SEQUENCE LISTING (8 sequences)

SEQ ID NO: 1 - DNA, 1410 bp, Homo sapiens
atgatcacagagggagcgcaggcccctcgattgttgctgccgccgctgctcctgctgctcaccctgccagccacaggctcagaccccgtgctctgcttcacccagtatgaagaatcctccggcaagtgcaagggcctcctggggggtggtgtcagcgtggaagactgctgtctcaacactgcctttgcctaccagaaacgtagtggtgggctctgtcagccttgcaggtccccacgatggtccctgtggtccacatgggccccctgttcggtgacgtgctctgagggctcccagctgcggtaccggcgctgtgtgggctggaatgggcagtgctctggaaaggtggcacctgggaccctggagtggcagctccaggcctgtgaggaccagcagtgctgtcctgagatgggcggctggtctggctgggggccctgggagccttgctctgtcacctgctccaaagggacccggacccgcaggcgagcctgtaatcaccctgctcccaagtgtgggggccactgcccaggacaggcacaggaatcagaggcctgtgacacccagcaggtctgccccacacacggggcctgggccacctggggcccctggaccccctgctcagcctcctgccacggtggaccccacgaacctaaggagacacgaagccgcaagtgttctgcacctgagccctcccagaaacctcctgggaagccctgcccggggctagcctacgagcagcggaggtgcaccggcctgccaccctgcccagtggctgggggctgggggccttggggccctgtgagcccctgccctgtgacctgtggcctgggccagaccatggaacaacggacgtgcaatcaccctgtgccccagcatgggggccccttctgtgctggcgatgccacccggacccacatctgcaacacagctgtgccctgccctgtggatggggagtgggactcgtggggggagtggagcccctgtatccgacggaacatgaagtccatcagctgtcaagaaatcccgggccagcagtcacgcgggaggacctgcaggggccgcaagtttgacggacatcgatgtgccgggcaacagcaggatatccggcactgctacagcatccagcactgccccttgaaaggatcatggtcagagtggagtacctgggggctgtgcatgcccccctgtggacctaatcctacccgtgcccgccagcgcctctgcacacccttgctccccaagtacccgcccaccgtttccatggtcgaaggtcagggcgagaagaacgtgaccttctgggggagaccgctgccacggtgtgaggagctacaagggcagaagctggtggtggaggagaaacgaccatgtctacacgtgcctgcttgcaaagaccctgaggaagaggaactctaa

SEQ ID NO: 2 - DNA, 1395 bp, Mus musculus
atgcctgctgaaatgcaagcccctcagtggctcctgctgctactggttatcctgccagccacaggctcagaccctgtgctctgcttcacccagtatgaggagtcctctggcaggtgcaaaggcctacttgggagagacatcagggtagaagactgctgtctcaacgctgcctatgccttccaggagcatgatggtggcctctgtcaggcatgcaggtctccacaatggtcagcatggtccttatgggggccctgctcagttacatgttctgaggggtcccagctgcgacacaggcgctgtgtgggcagaggtggtcagtgctctgagaatgtggctcctggaactcttgagtggcagctacaggcctgtgaggaccagccatgctgtccagagatgggtggctggtctgagtggggaccctgggggccttgctctgtcacatgctccaaaggaacccagatccgtcaacgagtatgtgataatcctgctcctaagtgtgggggccactgcccaggagaggcccagcaatcacaggcctgtgacacccagaagacctgccccacacatggggcctgggcatcctggggcccctggagcccctgctcaggatcctgccttggtggtgctcaagaacctaaggagacacgaagccgctcatgttctgcaccagcaccttcacaccagccccctgggaaaccctgctcaggaccagcctatgagcataaggcctgcagtggcctaccaccttgcccagtggctggtggctgggggccatggagccctttgagcccctgctctgtgacttgtggcctgggccagaccctagagcaacggacatgtgatcaccctgcaccccgtcatgggggccccttttgtgctggtgatgccactcggaaccaaatgtgtaacaaagccgtaccttgccctgtaaacggggagtgggaggcctggggaaaatggagtgactgcagccggctgagaatgtccatcaactgtgaaggaaccccaggccagcagtcacgttcaaggagctgtggcggccgcaaatttaatgggaagccatgtgctggaaaactccaggatattcgacactgctataacatccataactgtatcatgaaaggttcatggtcacagtggagtacctggagtctgtgcacaccaccatgtagtcccaacgccacccgtgtccgccagcgcctctgcacacctttgctccccaagtacccgcctacagtttcaatggttgaaggtcagggtgagaagaatgttaccttctgggggactccacggccactgtgtgaagcgctacaggggcagaagctggtggtggaagagaaacggtcatgtctacatgtgcctgtctgcaaagacccagaagagaagaaaccctaa

SEQ ID NO: 3 - DNA, 1413 bp, Cavia porcellus
atgactgccccagtgcaggtccctcaatcgctgctgctgctgctgatgctgctgctcaccctgccggccacaggctcggacccagtgctctgcttttcccagtatgaagactcctcaagcaagtgcaaggacctcctggggaaagatgtcagtttggaggactgctgtctcaatgctgcttatgccttcaaggagcgtgacaatgggcactgccaggcatgcaggtccccacgatggtcaccgtggtcctcatgggccccctgctcagtgtcatgctctgaaggctcccagctgcgccaccgccgatgcataggctggggtgggcagtgctctgagaacaaggctcctggtacccttgaatggcagctgcaggcctgtgaggagcagcaatgctgtcctgagatgggcggctggtctaactgggaaccctgggggccttgcacggtcacttgctccaaaggaacccgaatccgccgtcgagtatgtaataaccctgcccccaagtgtggggggcactgcccaggagtggcccaggaatcggaagcctgcgacacccagcaggtctgccctacccatggcgcctggggtccctggggcccctggagttcgtgcttaagctcctgtcacggtggaccccacaaacctgtggagacacgaagccgcacatgttctgcccctgagccctccaagaatccccctggaaatccctgtccaggaacagcctatgagcagcagagctgtgctggtctgccaccctgcccagtggctgggggctggggaccatggggctctgtgagcccctgttctgtgacctgcggcctgggccagatcttggaacaacggaagtgcgataaccctgtgccccagcatggaggcagcttctgtactggtgatgacacccgtgcccacatctgcaacacagctgtgccttgccctgtggacggagagtgggagccctggggtgactggagtacttgcacccgcccgcacctgagcgccatccgctgtaaggaaatcgtaggccagcagacacgcgtaaggatctgcaagggccgaaagtttaatggccagcgctgccctgggaaacaccaggaaatccgacactgctataacatccaaaactgcatctttggagagaaaggctcatggtcacagtggactccctgggggctgtgtacacccccatgtggggccaaccccacccgcgtccgccagcgccgctgcatggcctcactccccaagttctcacccacagtttccgtcgtcgaaggccagggtgagaagaacgttaccttctgggggaaaccattggcgcagtgcgaggagctgcaagggcagaaggtgctgctggaggagaaacgaccatgtctacacgtgcctgcctgcaaagaccctgaggaggaagagccctaa

SEQ ID NO: 4 - DNA, 1398 bp, Cricetulus griseus
atgcccgttggaatgcaagcccctcagtggctcctgctcctattgcttatcctgccaaccacaggctcagaccctgtgctctgcttcacccagtatgaggagccctctggcaggtgcaaaggcctacttggaagagacatcagggtggaagattgctgtctcaacactgcctatgccttccaggaacatgatgggggcctctgtcagtcatgcaggtccccacaatggtcagcatggtcctcatgggggccctgctcagttacatgctctgaggggtcccagctgcgacacaggcgctgtgtgggtaggggtggacagtgctctgagaaggcggctcctggaactcttgagtggcagctacaggcctgtgaggatcagctatgctgtccagagatgggtggctggtctgagtggggaccctgggggccttgctctgtcacctgctccaaaggaacccagacccgtcaacgactatgtgataatcctgctcctaagtgtggaggccattgccccggagaggcccagcaatcacaggcctgtgacactcagaagatctgccccacacatggggcctgggcatcctggggcccctggagcgcctgctcaggatcatgccttggcggagctcaagaacctaaggagacacgaagccgctcatgttctgcaccagcaccttcacaccaaccccctgggaagccctgctcaggaacagcctatgagcatcggggctgtagtggcctaccaccttgcccagtggctggtggctggggaccatggggcccttcaagtccctgcccggtgacttgtggcctgggccagaccctagaacgacggacatgtgatcaccctgtaccccgtcatgggggccccttttgtgctggtgatgccactcggaaacacgtctgtaacacagccatgccttgccctgtaaatggagagtgggaggcctggggaaaatggagtcactgcagccgtgtgagaatgaagtccatcagctgtgatgaaatcccaggccagcagtcacgttcaaggagctgtggtggccgcaaatttgatgggcagccatgtactgggaaactccaagatattcgacactgctatgacatccataactgtgtcttgaaaggttcatggtcacagtggagtacctggggtctgtgcacaccaccatgtggacccaaccccacccgtgtccgtcagcgactctgcacacctttgctccccaagtactcgcctacagtttccatggttgaaggtcagggtgagaagaatgttaccttctgggggatcccacggccactgtgtgaggtgctacaggggcagaagctggtggtggaagagaaacggccatgtctacatgtgccttcctgcagagatccagaagagaagaaaccctaa

SEQ ID NO: 5 - DNA, 27 bp, Artificial Sequence
  primer
aagcttatgatcacagagggagcgcag

SEQ ID NO: 6 - DNA, 37 bp, Artificial Sequence
  primer
ctcgagagtagagttcctcttcctcagggtctttgca

SEQ ID NO: 7 - DNA, 28 bp, Artificial Sequence
  primer
aagcttatgcctgctgaaatgcaagccc

SEQ ID NO: 8 - DNA, 34 bp, Artificial Sequence
  primer
ctcgagagtagggtttcttctcttctgggtcttt

(19) United States
(12) Patent Application Publication
(10) Pub. No.: US 2015/0038420 A1
(43) Pub. Date: Feb. 5, 2015

(54) METHODS FOR TREATING PROPERDIN-RELATED DISEASES OR DISORDERS

(71) Applicants: Hans-Wilhelm Schwaeble, Mount Sorrel (GB); Mohammed Youssif Ibrahim Ali, Mansoura (EG); Dimitrios Gkountis, Denville, NJ (US)

(72) Inventors: Hans-Wilhelm Schwaeble, Mount Sorrel (GB); Mohammed Youssif Ibrahim Ali, Mansoura (EG); Dimitrios Gkountis, Denville, NJ (US)

(73) Assignee: THE MEDICINES COMPANY, Parsippany, NJ (US)

(21) Appl. No.: 14/342,005

(22) PCT Filed: Aug. 31, 2012
(86) PCT No.: PCT/US2012/053315

§ 371 Date: Oct. 10, 2014

Related U.S. Application Data
(60) Provisional application No. 61/530,588, filed on Sep. 2, 2011.

(51) Int. Cl. A61K 38/17

(52) U.S. Cl. CPC A61K 38/1722
USPC 514/13.8; 530/386; 514/15.3

(57) ABSTRACT

The present invention relates to methods for treating or preventing a properd related disease or disorder in a subject in need thereof, or for reducing mortality of a subject suffering from a properdin-related disease or disorder, comprising administering to the subject an effective amount of a pharmaceutical composition comprising a properdin protein. Also provided are related medicaments, pharmaceutical compositions, and methods for preparing the medicaments.

### CROSS-REFERENCE TO RELATED APPLICATIONS

This application claims the benefit of U.S. Provisional Application No. 61/530,588, filed on Sep. 2, 2011, the contents of which are incorporated by reference herein, in their entireties and for all purposes.

### BACKGROUND OF THE INVENTION

Complement is the first-line of immune defense by a host against a pathogen before the host generates a specific and adapted immune response. Complement is regulated by three distinct pathways, each of which is subject to a different set of activators: antibody-antigen complexes for the classical pathway; certain sugar moieties for the lectin pathway; and microbial surfaces for the alternative pathway. Each pathway leads to the assembly of C3 convertases that cleave the fluid phase protein C3 into opsonin C3b, the major effector molecule of complement.

Properdin optimizes the alternative pathway activation rates by binding and stabilizing the inherently labile C3 and C5 convertase complexes (C3bBb and C3bₙBb). The C3bBb complex, has a half life of only 1.5 min, which can be increased by 5-10 fold upon association with properdin. Inherited properdin deficiency is an X-linked disorder, and linked to various diseases or disorders. Approximately 50% of the patients suffer severe, fulminant bacterial infections, usually with Neisseria meningitidis of serogroups B, C, Y and W-135, with a fatality rate close to 75%. There are three phenotypic forms of inherited properdin deficiency: the complete absence of properdin (type I), the very low level (1-10% of normal) presence of properdin (type II), and the presence of a dysfunctional properdin in serum (type III).

Properdin has been considered as a target for anti-inflammatory intervention with therapeutic antibodies (e.g., after cardiac surgery). However, there remains a need for an effective method for treating or preventing properdin-related diseases or disorders, especially for reducing the high mortality rate induced by Neisseria meningitidis.

### SUMMARY OF THE INVENTION

The present invention relates to the use of a properdin protein for treating or prevention properdin-related diseases or disorders, especially for protecting against Neisseria meningitidis, and pharmaceutical compositions or medicaments comprising the properdin protein.

A method for treating or preventing a properdin-related disease or disorder in a subject in need thereof is provided. Also provided is a method for reducing mortality of a subject suffering from a properdin-related disease or disorder. These methods comprise administering to the subject an effective amount of a pharmaceutical composition comprising a properdin protein. The mortality may be reduced by at least about 10%, 20%, 30%, 40%, 50%, 60%, 70%, 80%, 90%, or 100%, preferably by at least about 50%, more preferably by at least about 60%. The effective amount of the pharmaceutical composition is selected to achieve a target serum concentration of properdin in the subject in the range of about 0.1-500 μg/ml, preferably about 0.5-250 μg/ml, more preferably about 1-100 μg/ml, most preferably about 100 μg/ml.

The properdin-related disease or disorder may be selected from the group consisting of a complement deficiency, infectious disease, platelet adhesion disorder, cancer, and inflammation. The meningitis may be bacterial meningitis, viral meningitis, fungal meningitis, parasitic meningitis, or noninfectious meningitis.

The bacterial meningitis may be caused by a bacterium selected from the group consisting of Streptococcus pneumoniae, Neisseria meningitidis, Haemophilus influenzae, Listeria monocytogenes, Group B streptococci, Escherichia coli, and Mycobacteria. The Neisseria meningitidis may be serogroup A, B, C, W135, X or Y, preferably serogroup B, C, Y or W135.

The viral meningitis may be caused by a virus selected from the group consisting of enteroviruses, herpesvirus, arbovirus, mumps virus, glandular fever virus, human immunodeficiency virus (HIV) and lymphocytic choriomeningitis virus (LCMV).

The herpesvirus may be selected from the group consisting of Epstein-Barr virus, herpes simplex virus, varicella-zoster virus, cytomegalovirus (CMV), measles, and influenza. The arbovirus may be West Nile virus or La Crosse virus.

The parasitic meningitis may be caused by a parasite selected from the group consisting of Angiostrongylus cantonensis, Gnathostoma spinigerum, and Schistosoma.

The noninfectious meningitis may be caused by carcinomatosis, cancer, a head injury, a brain surgery, a birth defect of the skull, or a medication. The medication may be a nonsteroidal anti-inflammatory drug or antibiotic. The nonsteroidal anti-inflammatory drug may be ibuprofen or naproxen. The antibiotic may be trimethoprim, sulfamethoxazole, or a combination thereof.

The subject may be a male or female. The subject may be a mammal, preferably a mouse or human, more preferably a human. The subject may be a premature baby, newborn, child or adult. The subject is up to 3 months old, under 5 years old, or over 50 years old.

The subject may have a type I, II or III inherited properdin deficiency. The subject may have suffered from a properdin-related disease or disorder, septicemia, malaria, platelet adhesion disorder, cancer, inflammation or metastasis inflammation.

The subject may have suffered from meningitis, preferably caused by Neisseria meningitidis, more preferably caused by serogroup A, B, C, W135, X or Y Neisseria meningitidis, most preferably caused by serogroup B, C, Y or W135 Neisseria meningitidis. The subject may have suffered from cysticercosis, toxocariasis, baylisascariasis, or paragonimiasis.

The pharmaceutical composition may comprise an effective amount of a properdin protein. The effective amount of the properdin protein is selected to achieve a target serum concentration of properdin in the subject in the range of about 0.1-500 μg/ml, preferably about 0.5-250 μg/ml, more preferably about 1-100 μg/ml, most preferably about 100 μg/ml. The pharmaceutical composition may comprise about 0.01-20,000 μg, preferably about 0.1-1000 μg, more preferably 0.5-500 μg, most preferably about 100 μg of the properdin protein. The pharmaceutical composition may comprise about 0.01-20,000 μg/ml, preferably about 0.1-1000 μg/ml, more preferably 0.5-500 μg/ml, most preferably about 100 μg/ml of the properdin protein. The pharmaceutical composition may further comprise a pharmaceutically acceptable carrier or diluent. The pharmaceutical composition may have a pH of 5.6-10.0.

The properdin protein may be a natural or recombinant protein. The properdin protein is prepared from human plasma. The properdin protein may be purified or not purified. The properdin may be a monomer, dimer, trimer, tetramer, or a combination thereof, preferably a dimer, trimer, tetramer, or a combination thereof, more preferably a tetramer.

The pharmaceutical composition may be formulated for oral, sublingual, intranasal, intraocular, rectal, transdermal, mucosal, topical or parenteral administration. The pharmaceutical composition may be administered to the subject by an intraperitoneal injection.

The method according to the present invention may further comprise administering to the subject an antibiotic, steroid, antiviral drug or analgesic. The antibiotic may be benzylpenicillin, ceftriaxone or vancomycin. The steroid may be a corticosteroid. The corticosteroid may be dexamethasone. The antiviral drug may be acyclovir.

For each of the methods described herein, a medicament comprising an effective amount of a properdin protein is provided. The medicament is useful for treating or preventing a properdin-related disease or disorder in a subject, and/or reducing mortality of a subject suffering from a properdin-related disease or disorder. The effective amount of the properdin protein is selected to achieve a target serum concentration of properdin in the subject in the range of about 0.1-500 μg/ml, preferably about 0.5-250 μg/ml, more preferably about 1-100 μg/ml, most preferably about 100 μg/ml. The medicament may comprise about 0.01-20,000 μg, preferably about 0.1-1000 μg, more preferably 0.5-500 μg, most preferably about 100 μg of the properdin protein. The medicament may comprise about 0.01-20,000 μg/ml, preferably about 0.1-1000 μg/ml, more preferably 0.5-500 μg/ml, most preferably about 100 μg/ml of the properdin protein. The medicament may further comprise a pharmaceutically acceptable carrier or diluent. The medicament may have a pH of 5,6-10.0.

A method for preparing a medicament useful for treating or preventing a properdin-related disease or disorder in a subject, and/or reducing mortality of a subject suffering from a properdin-related disease or disorder, is provided. The method may comprise admixing a properdin protein with a pharmaceutically acceptable carrier or diluent. The method may further comprise adding one or more agents selected from the group consisting of an antibiotic, steroid, antiviral drug, and analgesic.

### BRIEF DESCRIPTION OF THE DRAWINGS

FIG. 1 shows DNA sequences for (A) human properdin (SEQ ID NO: 1), (B) murine properdin (SEQ ID NO: 2), (C) guinea pig properdin (SEQ ID NO: 3), and (D) rat properdin (SEQ ID NO: 4).

FIG. 2A shows agaose gel electrophoresis image of PCR product of human properdin on 1% gel. The PCR product shows a single band at 1005 by against 1 kb

DNA ladder. The full length human properdin coding sequence was ligated into the 5.2 kb expression vector pSectag2/hygroB. FIG. 2B shows two positive clones after digestion of the insert from the expression vector with Xhol and Hind III restriction enzymes.

FIG. 3 shows SDS-PAGE of human properdin after purification. Lanes 1:

Protein ladder; 2: culture supernatant (S); 3: column flow through (F); 4: Column wash (W); 5: El fraction under reducing condition; 6: E2 fraction under reducing condition; 7: E3 fraction under reducing condition; 8: El fraction under non-reducing condition; 9: E2 fraction under non-reducing condition; 10: E3 fraction under non-reducing condition.

FIG. 4 shows SDS-PAGE of mouse properdin after purification under reducing condition (R) and non-reducing conditions (NR). The mouse properdin protein gives a band at 57 kD.

FIG. 5 shows Western blot analysis of human properdin under reducing condition (R) and non-reducing condition (NR) using anti-hProperdin Ab.

FIG. 6 shows Western blot analysis of mouse properdin under reducing condition (R) and non-reducing condition (NR) using anti-hProperdin Ab.

FIG. 7 shows that pre-treatment of wild type C57BL6 mice with a properdin protein (100 μg/mouse) improved the survival rate of the mice following an intraperitoneal (i.p.) injection with a high dose (8×10⁶ cfu/100 μl) of N. meninigitidis serogroup B-MC58. The infective dose was supplemented by the addition of iron to a final concentration of 400 mg/kg mice in order to support bacterial growth in the mouse.

### DETAILED DESCRIPTION OF THE INVENTION

The present invention is based on the discovery that pre-treatment with a properdin protein protected mice against Neisseria meningitis. In particular, the properdin protein reduced the Neisseria meningitis induced mortality rate in infected mice from 100% to 40%.

The terms “protein” and “polypeptide” are used herein interchangeably, and refer to a polymer of amino acid residues with no limitation with respect to the minimum length of the polymer. The definition includes both full-length proteins and fragments thereof, as well as modifications thereof (e.g., glycosylation, phosphorylation, deletions, additions and substitutions).

The term “derived from” used herein refers to the origin or source, and may include naturally occurring, recombinant, unpurified or purified molecules.

The term “fragment” of a protein as used herein refers to a polypeptide having an amino acid sequence that is the same as a part, but not all, of the amino acid sequence of the protein.

The term “variant” of a protein as used herein refers to a polypeptide having an amino acid sequence that is the same as the amino acid sequence of the protein except having at least one amino acid modified, for example, deleted, inserted, or replaced. The variant may have an amino acid sequence at least about 80%, 90%, 95%, or 99%, preferably at least about 90%, more preferably at least about 95%, identical to the amino acid sequence of the protein.

Properdin, or Complement Factor P (CFP), is a glycoprotein circulating as dimers, trimers and tetramers of a single chain glycoprotein in blood serum. Each subunit is composed mainly of 6 thrombospondin-like repeat domains. Properdin stabilizes the inherently labile C3 and C5 convertase complexes (C3bBb and C3bₙBb), and thus acts as a positive regulator of the alternative pathway of complement. The functional activity of the polymers increases with their size with the tetramer being 10 times as active as the dimer. Glycosylation has no effect on secretion, polymer formation and properdin functional activity (Farries and Atkinson, 1989). Properdin is present in plasma at fairly constant low concentrations (5-15 μg/ml). (Schwaeble and Reid, 1999, Immunology Today 20(1):17-21). It has been reported that addition of purified properdin can restore alternative pathway activation in properdin-deficient sera in a dose-dependent manner with complete restoration at 50% of the normal serum concentration. (Schwaeble and Reid, 1999, Immunology Today 20(1):17-21).

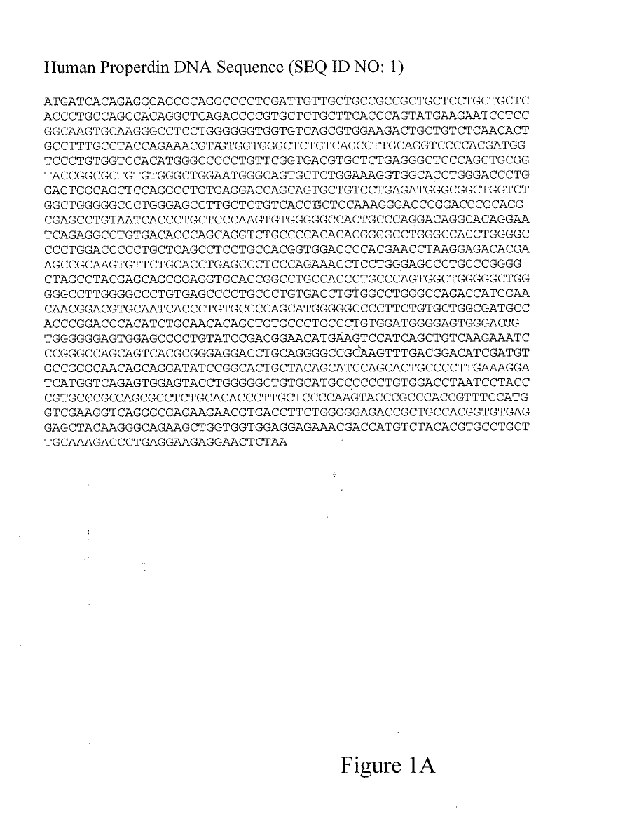

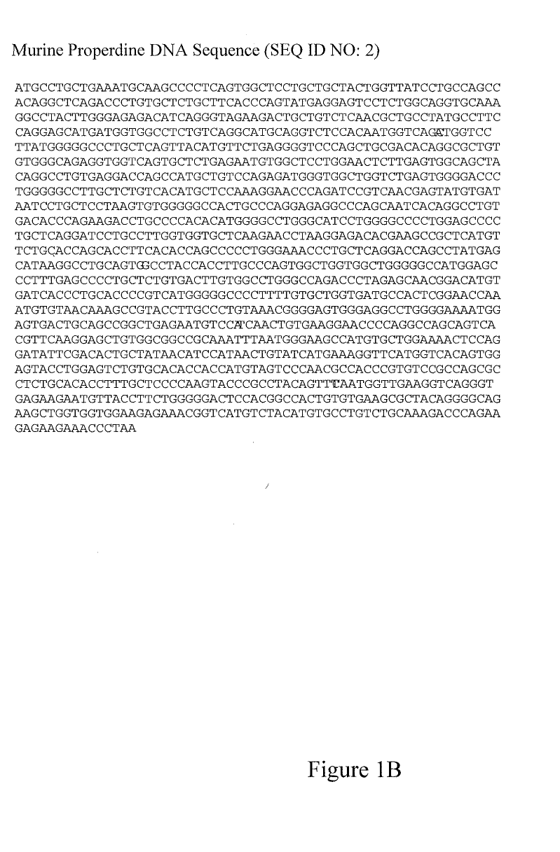

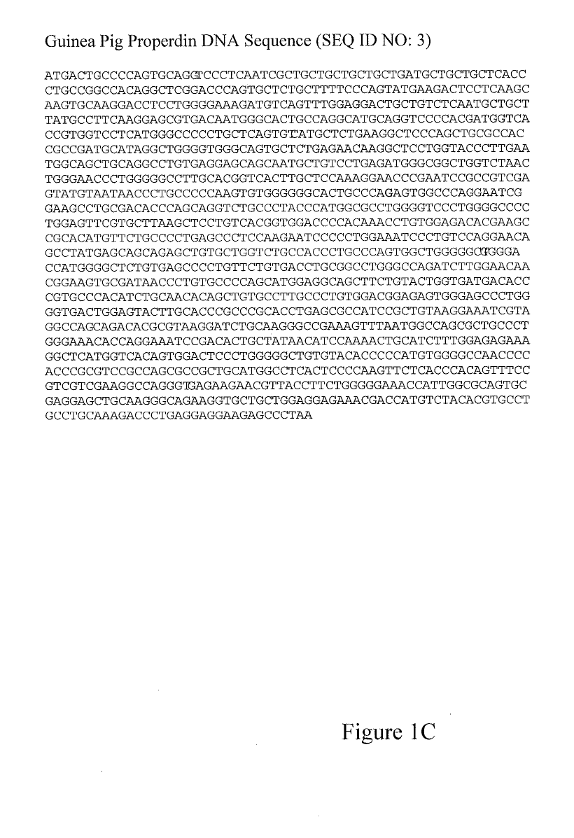

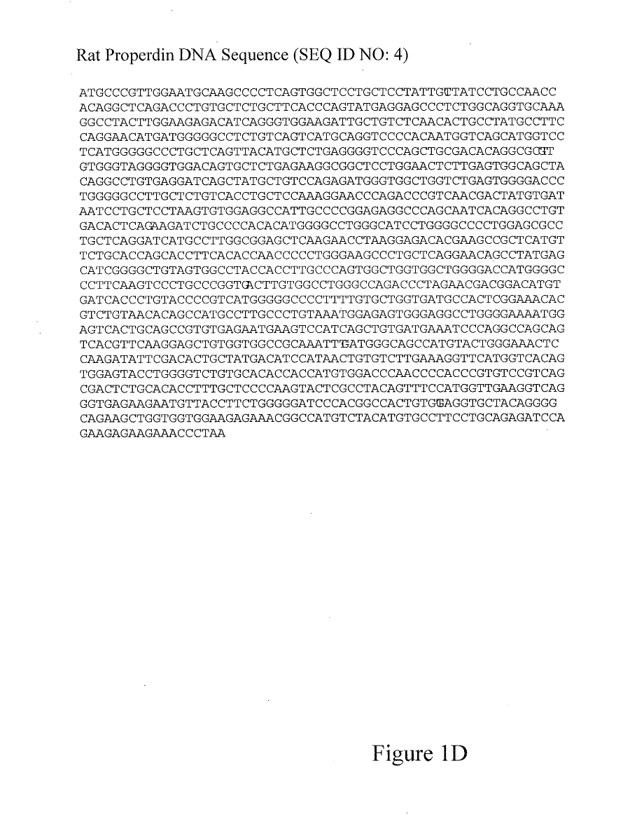

The properdin protein and gene sequences are known in the art. For example, the amino acid sequences of human, mouse, guinea pig, and rat properdin can be found in the GenBank database, for example, P27918 for human properdin, P11680 for murine properdin, Q64181 for guinea pig properdin, BOBNN4 for rat properdin. The exemplary cDNA sequences for human, mouse, guinea pig, and rat properdin are shown in FIG. 1. Amino acid sequence alignment of human properdin with mouse and rat properdin showed an approximate degree of 78% similarity while human properdin shows an approximate degree of 75% similarity with the derived guinea pig amino acid sequence properdin. Natural properdin protein may be purified from normal serum. Recombinant properdin protein may be expressed and purified using conventional techniques.

The present invention provides various methods, including a method for treating or preventing a properdin-related disease or disorder in a subject in need thereof, and a method for reducing mortality of a subject suffering from a properdin-related disease or disorder. These methods comprise administering to the subject an effective amount of a pharmaceutical composition comprising a properdin protein. The mortality may be reduced by at least about 10%, 20%, 30%, 40%, 50%, 60%, 70%, 80%, 90%, or 100%, preferably by at least about 50%, more preferably by at least about 60%.

The term “a properdin protein” as used herein refers to a full length properdin, or a functional fragment or variant thereof, in the form of a monomer, dimer, trimer, or tetramer. The properdin protein may be a natural or recombinant protein, purified or unpurified. The properdin protein may be derived from a mammal, preferably from normal serum of a human, mouse, guinea pig or rat, more preferably from normal human serum. The properdin protein is also available commercially (Complement Technology; Quidel). A properdin fragment or variant is functional where the fragment or variant is capable of binding the C3Bb complex.

The term “properdin-related disease or disorder” as used herein refers to a disease or disorder linked to properdin. Examples of properdin-related diseases or disorders include complement deficiency, infectious disease, platelet adhesion disorder, cancer, and inflammation. Other examples of properdin-related diseases or disorders include hypocomplementemia, afibrinogenemia, acute poststreptococcal glomerulonephritis, agammaglobulinemia, agammaglobulinemia swiss type, glomerular disease, ametropia, wiskott-aldrich syndrome, IgA glomerulonephritis, protein c deficiency, nephritis, chronic granulomatous disease, proteinuria, septic shock, hemolysis, systemic infection, septicemia, immunologic deficiency syndrome, systemic lupus erythematosus, thrombosis, diabetes mellitus, and necrosis. The infectious disease may be meningitis or malaria. The inflammation may be metastasis inflammation.

The properdin-related disease or disorder is preferably meningitis. The meningitis may be bacterial meningitis, viral meningitis, fungal meningitis, parasitic meningitis, or noninfectious meningitis.

The bacterial meningitis may be caused by a bacterium selected from the group consisting of Streptococcus pneumoniae, Neisseria meningitidis, Haemophilus influenzae, Listeria monocytogenes, Group B streptococci, Escherichia coli, and Mycobacteria. The Neisseria meningitidis may be serogroup A, B, C, W135, X or Y, preferably serogroup B, C, Y or W135.

The viral meningitis may be caused by a virus selected from the group consisting of enteroviruses, herpesvirus, arbovirus, mumps virus, glandular fever virus, human immunodeficiency virus (HIV) and lymphocytic choriomeningitis virus (LCMV). The herpesvirus may be Epstein-Barr virus, herpes simplex virus, varicella-zoster virus, cytomegalovirus (CMV), measles, or influenza. The arbovirus may be West Nile virus or La Crosse virus.

The noninfectious meningitis may be caused by carcinomatosis, cancer, a head injury, a brain surgery, a birth defect of the skull, or a medication. The medication may be a nonsteroidal anti-inflammatory drug or antibiotic. The nonsteroidal anti-inflammatory drug may be ibuprofen or naproxen. The antibiotic may be trimethoprim, sulfamethoxazole, or a combination thereof.

The parasitic meningitis may be caused by a parasite selected from the group consisting of Angiostrongylus cantonensis, Gnathostoma spinigerum, and Schistosoma.

The subject is a mammal, for example, a mouse, rat, dog, pig, or human, preferably a human. The subject may be male or female. The subject is a premature baby, newborn, child or adult. The subject may be up to 3 months old, under 5 years old, or over 50 years old.

The subject may have a type I, II or III inherited properdin deficiency. The subject may have suffered from a properdin-related disease or disorder. The subject may have suffered from septicemia, malaria, platelet adhesion disorder, cancer or inflammation. The inflammation may be metastasis inflammation. The subject may have suffered from meningitis, preferably caused by Neisseria meningitidis, more preferably serogroup A, B, C, W135, X or Y Neisseria meningitidis, most preferably serogroup B, C, Y or W135 Neisseria meningitidis. The subject may have suffered from a condition selected from the group consisting of cysticercosis, toxocariasis, baylisascariasis, and paragonimiasis.

The term “an effective amount” refers to an amount of a pharmaceutical composition comprising a properdin protein required to achieve a stated goal (e.g., treating or preventing properdin-related disease or disorder in a subject in need thereof, and/or reducing mortality of a subject suffering from a properdin-related disease or disorder). The effective amount of the pharmaceutical composition may be selected to achieve a target serum concentration of properdin in the subject in the range of about 0.1-500 μg/ml, preferably about 0.5-250 μg/ml, more preferably about 1-100 μg/ml, most preferably about 100 μg/ml, with one or multiple doses of the composition. The effective amount of the pharmaceutical composition comprising a properdin protein may vary depending upon the stated goals, the physical characteristics of the subject, the nature and severity of the properdin-related disease or disorder, the existence of related or unrelated medical conditions, the nature of the properdin protein, the composition comprising the properdin protein, the means of administering the composition to the subject, and the administration route. A specific dose for a given subject may generally be set by the judgment of a physician. The pharmaceutical composition may be administered to the subject in one or multiple doses. Each does may be at about 0.01-5000 mg/kg, preferably about 0.1-1000 mg/kg, more preferably about 1-500 mg/kg.

The pharmaceutical composition may comprise an effective amount of a properdin protein. The effective amount of the properdin protein may be selected to achieve a target serum concentration of properdin in the subject in the range of about 0.1-500 μg/ml, preferably about 0.5-250 μg/ml, more preferably about 1-100 μg/ml, most preferably about 100 μg/ml. The pharmaceutical composition may comprise about 0.01-20,000 μg, preferably about 0.1-1000 μg, more preferably 0.5-500 μg, most preferably about 100 μg of the properdin protein. The pharmaceutical composition may comprise about 0.01-20,000 μg/ml, preferably about 0.1-1000 μg/ml, more preferably 0.5-500 μg/ml, most preferably about 100 μg/ml of the properdin protein. The pharmaceutical composition may further comprise a pharmaceutically acceptable carrier or diluent. Carriers, diluents and excipients suitable in the pharmaceutical composition are well known in the art.

The pharmaceutical composition may have a pH of about 5.6-10.0, preferably about 6.0-8.8, more preferably about 6.5-8.0. For example, the pH may be about 6.2, 6.5, 6.75, 7.0, or 7.5.

The pharmaceutical compositions of the present invention may be formulated for oral, sublingual, intranasal, intraocular, rectal, transdermal, mucosal, topical or parenteral administration. Parenteral administration may include intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids) injection or infusion, preferably intraperitoneal (i.p.) injection in mouse and intravenous (i.v.) in human. Any device suitable for parenteral injection or infusion of drug formulations may be used for such administration. For example, the pharmaceutical composition may be contained in a sterile pre-filled syringe.

The method of the present invention may further comprise administering to the subject an antibiotic (e.g., benzylpenicillin, ceftriaxone or vancomycin), steroid such as corticosteroid (e.g., dexamethasone), antiviral drug (e.g., acyclovir), or analgesic.

In some embodiments, medicaments comprising an effective amount of a properdin protein are provided. The medicaments are useful for treating or preventing a properdin-related disease or disorder in a subject, or reducing mortality of a subject suffering from a properdin-related disease or disorder.

The medicament may comprise an effective amount of a properdin protein. The effective amount of the properdin protein may be selected to achieve a target serum concentration of properdin in the subject in the range of about 0.1-500 μg/ml, preferably about 0.5-250 μg/ml, more preferably about 1-100 μg/ml, most preferably about 100 μg/ml. The medicament may comprise about 0.01-20,000 μg, preferably about 0.1-1000 μg, more preferably 0.5-500 μg, most preferably about 100 μg of the properdin protein. The medicament may comprise about 0.01-20,000 μg/ml, preferably about 0.1-1000 μg/ml, more preferably 0.5-500 μg/ml, most preferably about 100 μg/ml of the properdin protein. The medicament may further comprise a pharmaceutically acceptable carrier or diluent. Carriers, diluents and excipients suitable in the pharmaceutical composition are well known in the art.

In a medicament according to the present invention, the properdin protein may be a natural or recombinant protein. The properdin may be purified or not purified. The properdin may be prepared from human plasma. The properdin may be a monomer, dimer, trimer, tetramer, or a combination thereof, preferably a dimer, trimer, tetramer, or a combination thereof, more preferably a tetramer.

The medicament may have a pH of about 5.6-10.0, preferably about 6.0-8.8, more preferably about 6.5-8.0. For example, the pH may be about 6.2, 6.5, 6.75, 7.0, or 7.5.

The medicament may further comprise an antibiotic (e.g., benzylpenicillin, ceftriaxone or vancomycin), steroid such as corticosteroid (e.g., dexamethasone), antiviral drug (e.g., acyclovir), or analgesic.

In some other embodiments, methods for preparing the medicaments useful for treating or preventing a properdin-related disease or disorder in a subject, or useful for reducing mortality of a subject suffering from a properdin-related disease or disorder, are provided. The preparation methods may comprise admixing a properdin protein with a pharmaceutically acceptable carrier or diluent. The methods may further comprise adding one or more agents selected from the group consisting of an antibiotic, steroid, antiviral drug, and analgesic. The antibiotic may be benzylpenicillin, ceftriaxone or vancomycin. The steroid may be a corticosteroid (e.g., dexamethasone). The antiviral drug may be acyclovir. The properdin protein is a natural or recombinant protein. The properdin protein may be prepared from human plasma. The properdin protein may be purified or not purified. The properdin may be a monomer, dimer, trimer, tetramer, or a combination thereof, preferably a dimer, trimer, tetramer, or a combination thereof, more preferably a tetramer.

### EXAMPLE 1

### Engineering of Human and Murine Properdin

A. Cloning Human and Murine Properdin ORF

The coding sequences for murine properdin (GenBank Accession No. P11680) and human properdin (Genbank Accession No. P27918) were amplified using the corresponding primers, (a) primers hfP_Hind_PsecB_F and hfP_Xhol_PsecB_R for human properdin, and (b) primers mfP_HindIII_F and mfP_Xhol_R for murine properdin:

| | |
|---|---|
| hfP_Hind_PsecB_F | AAG CTT ATG ATC ACA GAG GGA GCG |
| | CAG |
| hfP_Xho1_PsecB_R | CTC GAG AGT AGA GTT CCT CTT CCT |
| | CAG GGT CTT TGC A |
| mfP_HindIII_F | AAG CTT ATG CCT GCT GAA ATG CAA |
| | GCC C |
| mfP_Xho1_R | CTC GAG AGT AGG GTT TCT TCT CTT |
| | CTG GGT CTT T |

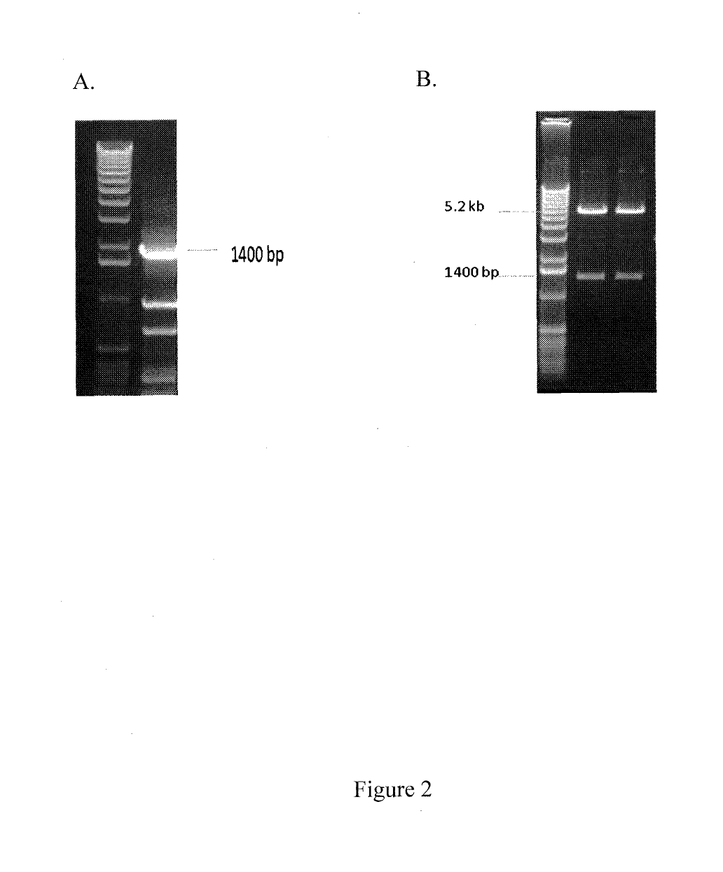

In the reverse primer, the TAA stop codon was mutated into TAC code of Tyrosine, by this mutation the properdin ORF becomes in-frame with the 6-hist tag residues in the expression vector Psectag2/hygroB. The human properdin PCR product shows a single band at 1005 by (FIG. 2A).

For cloning, pGEM-T Easy vector (Promega) was used. The pGEM-T easy vector map showing the multiple cloning site is available at Promega official website. This is an open vector with 3′ terminal thymidine at both ends. The presence of these single 3′-T overhangs located at the insertion site are known to improve the efficiency of ligation of a PCR product into the plasmids, by preventing self-ligation of the vector. In brief, PCR product ligation into pGEM-T Easy vector (50 ng/ul) was done following the conventional ligation reaction as represented in the following ratio (Sambrook et al., 1989, Molecular Cloning: A Laboratory Manual (Second Edition):

$$ng insert of DNA = \frac{( ng vector DNA ) \times ( kb size of the insert DNA )}{( Kb size of the vector )} \times \text{?} \begin{matrix}molar ratio \\ vector\end{matrix}$$

$$\text{?} \text{indicates text missing or illegible when filed}$$

The final volume of ligation was 10 μl in de-ionised water containing 50 ng of vector DNA, x ng of insert (the calculation is according for above equation), 1 μl of T₄ DNA ligase (New England Biolabs.) and 1 μl (10×) T₄ DNA ligase buffer. The reaction mixture was kept at 4° C. overnight.

The DNA construct in the pGEM-T easy was digested with Xho1 and HindIII restriction enzymes, and the full length human properdin coding sequence of 1400 by was ligated into the 5.2 kb expression vector pSecTag2/hygroB (Invitrogen), which was previously digested with the same restriction enzymes (FIG. 2B). The pSectag2/hygroB vector map showing the multiple cloning site (Invitrogen, official website).

B. Expression and Purification of Human and Murine Properdin Protein

Human and murine protein were expressed in Chinese Hamster Ovary (CHO-K1) cell line. The cell line was routinely cultured in F12 nutrient mixture (Ham) and

Glutamax medium (GIBCO). The media were supplemented with 10% of heat inactivated foetal calf serum (Harlan) and 100 U of Penicillin/Streptomycin mixture (GIBCO) per ml of the medium. The cells were maintained in a 5% CO₂ incubator at 37° C. When the cells were 90% confluent, they were washed twice in 1× PBS (Oxoid). For cell detachment by trypsinization, 1× trypsin -EDTA solution (GIBCO) was added to the flask, and the cells were split to be cultured in a new flask in the presence of pre-warmed fresh serum-supplemented medium.

CHO-K1 cells were grown in 6-well plates supplemented with 3 ml of serum medium per well. Once they had reached 80% confluency, cells were transfected using GeneJuice reagent (Novagen) according to the manufacturer's protocol. Briefly, 3 μl of GeneJuice was mixed in a sterile microcentrifuge tube with 100 μl of serum-free RPMI 1640 medium using a vortex and incubated at room temperature. After 5 minutes of incubation, 1 μg of the DNA was added into the reaction mixture and mixed by pipetting. The reaction mixture was incubated for 15 minutes at room temperature. The entire transfection mixture was added drop-wise to CHO-K1 cells with gentle rocking to allow even distribution of the transfection reagent and DNA construct. After transfection, cells were incubated at 37° C. (5% CO2). After 48 hrs, cells expressing

Properdin were selected by addition of fresh serum medium containing hygromycin B (300 μg/ml) (Sigma).

Once the transfected cells in the 6-well plate had become 70-90% confluent, cells were washed twice with PBS, trypsinized and diluted to 10 cell/ml in the selection medium. After cell re-suspension, 100 μl of media containing 1 cell was added to each well of the 96 well plate and incubated at 37° C. (5% CO2) with medium change every 3 days for clonal selection of Properdin producing clones.

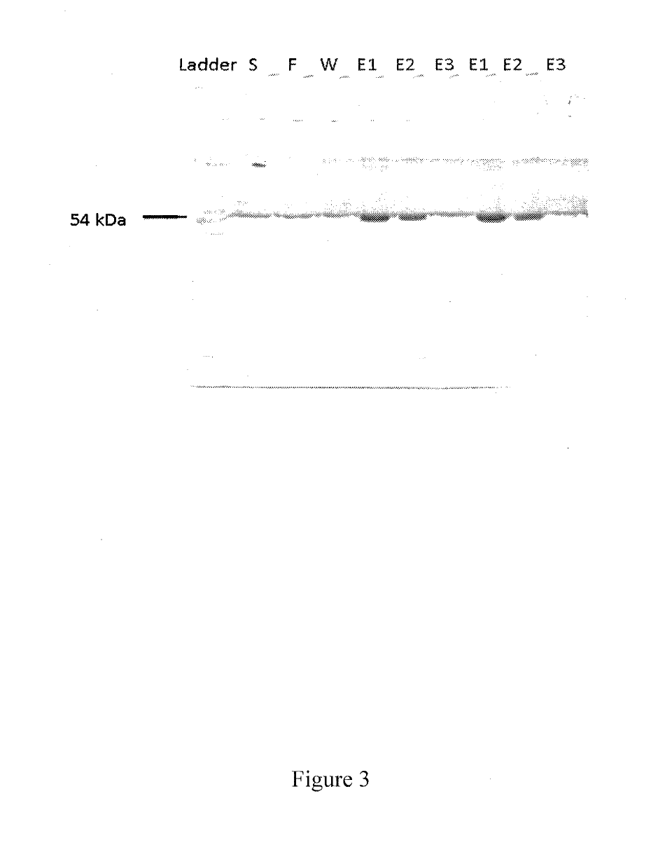

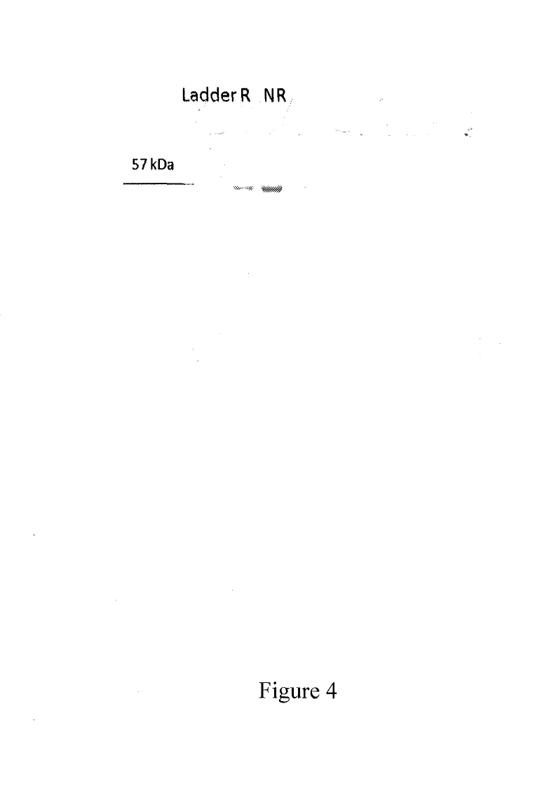

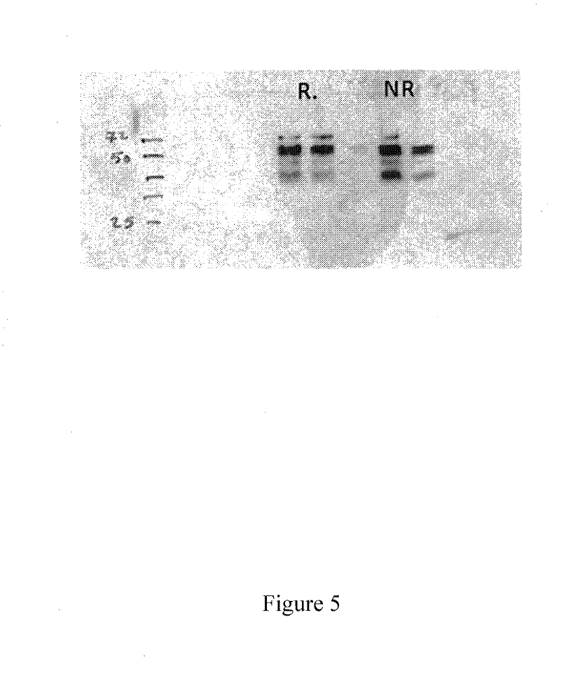

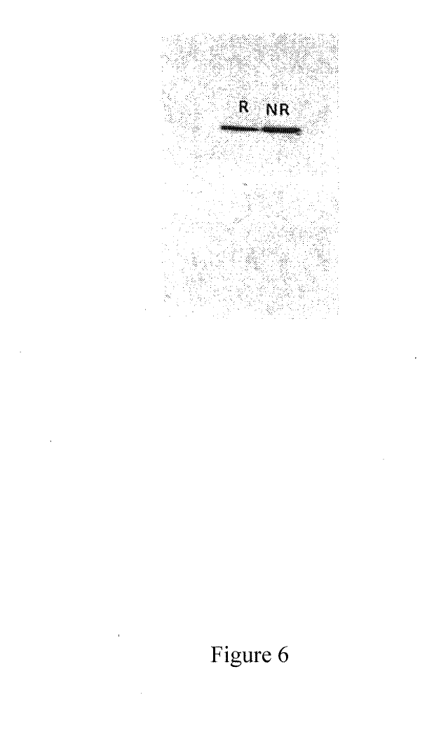

Following large scale protein expression by cellular transfection, protein purification was carried as follows: The transfected clones were allowed to grow in F12 Ham medium (Invitrogen) in three large flasks. The medium was supplemented with 10% fetal calf serum (Harlan), 100 u/ml penicillin/Streptomycin (GIBCO) and 300 ug/ml of Hygromycin B. After the cells reached 60% confluency, they were washed 3 times with PBS. Following the last wash, the growing medium was replaced with Chinese hamster ovary (CHO) serum-free II medium (Invitrogen) supplemented with 100 u/ml Penicillin/streptomycin (GIBCO) and 300 ug/ml of Hygromycin B. when the cells had been incubated for 72 hours, the medium was harvested. In order to eliminate cell debris, the medium was centriguged at 3000 xg for 10 minutes, Next, to purify Properdin, 200 ml of cell supernatant was diluted with an equal volume of 2× loading buffer (phosphate buffer pH 7.4, containing 100 mM NaCl and 5 mM imidazole) and loaded on HisGravi Trap column (GE Healthcare). The loaded sample was allowed to flow by the force of gravity through the column. The column was next washed with 20 ml of washing buffer (phosphate buffer pH 7.4, containing 100 mM NaCl and 25 mM imidazole), and the protein was eluted by elution buffer (phosphate buffer pH 7.4, 100 mM NaCl and 500 mM imidazole) in one ml fractions. Next, the protein fractions were loaded on SDS-PAGE (Sodium Dodecyl Sulfate Polyacrylamide Gel Electrophoresis) (FIG. 3 for human properdin; FIG. 4 for mouse properdin) followed by western blotting (FIG. 5 for human properdin; FIG. 6 for mouse properdin) to check protein purity, size and expression.

### EXAMPLE 2

### Properdin Reduces Neisseria meningitidis Induced Mortality

An infection study was carried out to evaluate whether pre-treatment with properdin would protect mice against Neisseria meningitidis. As described in Example 1, a full length recombinant mouse properdin protein was expressed in a CHO-k1 cell line having the properdin full length gene sequence (European Molecular Biological Laboratory/GenBank accession number X12905) in the pSectag2/hygroB mammalian expression vector. Six (6) His tag residues were added at the C-terminus to facilitate purification of the recombinant protein. The recombinant properdin was purified by affinity chromatography on Ni column binding to the C-terminal 6 His tag residues.

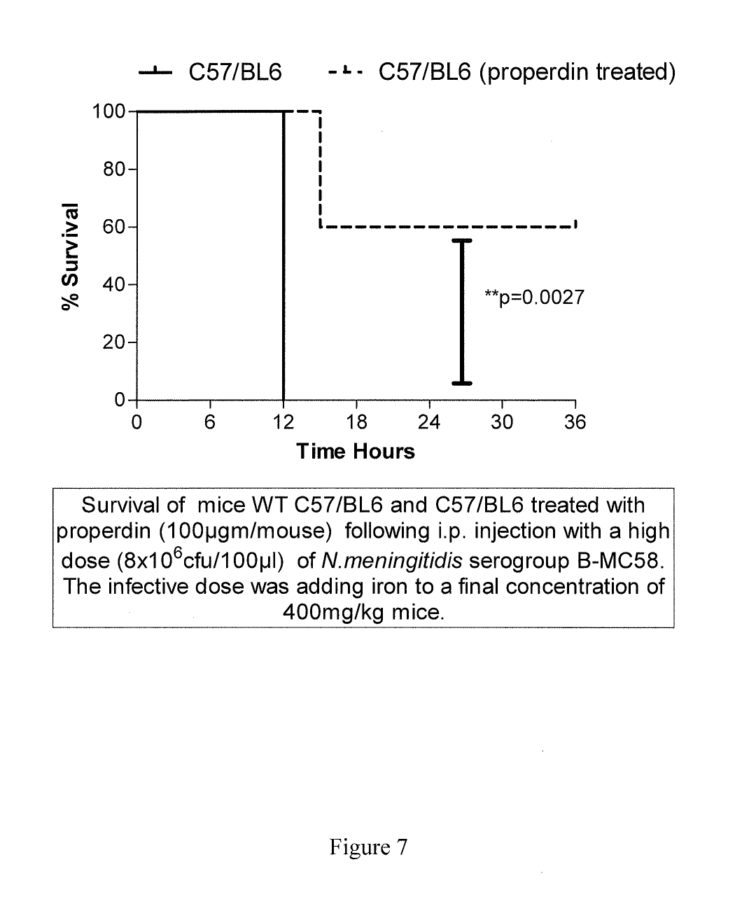

Two groups of wild type C57BL6 mice (n=5 each) were compared against each other in the infection study. Twelve (12) hours before infection, both groups were given iron-dextran (400 mg/kg) via i.p. infection. On the following day, one group (test group) was injected with the purified recombinant properdin protein at 100 μg per mouse (i.p). Six (6) hours after properdin injection, both groups were infected with 8×10⁶ (i.p.) of N. meninigitidis serogroup B-MC58, and mice were monitored. The survival rate for the control group (C57/BL6) dropped to zero 12 hours after the infection while the survival rate for the test group (properdin treated C57/BL6) dropped to 60% at 15 hours and remained the same until the end of the study (up to 36 hours) (FIG. 7).

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

All documents, books, manuals, papers, patents, published patent applications, guides, abstracts, and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A composition comprising an effective amount of a properdin protein useful for treating or preventing a properdin-related disease or disorder in a subject.

2. A composition comprising an effective amount of a properdin protein useful for reducing mortality of a subject suffering from a properdin-related disease or disorder.

3. The composition of claim 1, wherein the medicament comprises 0.01-20,000 μg of the properdin protein.

4. The composition of claim 1, wherein the medicament comprises 0.01-20,000 μg/ml of the properdin protein.

5. The composition of claim 1, wherein the effective amount of the properdin protein is selected to achieve a target serum concentration of properdin in the subject in the range of 1-100 μg/ml.

6. The composition of claim 1, wherein the medicament further comprises a pharmaceutically acceptable carrier or diluent.

7. The composition of claim 1, wherein the medicament has a pH of 5.6-10.0.

8. The composition of claim 1, wherein the properdin protein is a natural protein.

9. The composition of claim 1, wherein the properdin protein is a recombinant protein.

10. The composition of claim 1, wherein the properdin protein is prepared from human plasma.

11. A method for treating or preventing a properdin-related disease or disorder in a subject in need thereof, comprising administering to the subject an effective amount of a pharmaceutical composition comprising a properdin protein.

12. (canceled)

13. The method of claim 11, wherein the properdin-related disease or disorder is selected from the group consisting of a complement deficiency, infectious disease, platelet adhesion disorder, cancer, and inflammation.

14. The method of claim 11, wherein the properdin-related disease or disorder is meningitis.

15. The method of claim 14, wherein the meningitis is bacterial meningitis, viral meningitis, fungal meningitis, parasitic meningitis, or noninfectious meningitis.

16. The method of claim 15, wherein the bacterial meningitis is caused by a bacterium selected from the group consisting of Streptococcus pneumoniae, Neisseria meningitidis, Haemophilus influenzae, Listeria monocytogenes, Group B streptococci, Escherichia coli, and Mycobacteria.

17. The method of claim 16, wherein the Neisseria meningitidis is serogroup A, B, C, W135, X or Y.

18. The method of claim 11, wherein the pharmaceutical composition comprises 0.01-20,000 μg of the properdin protein.

19. The method of claim 11, wherein the pharmaceutical composition comprises 0.01-20,000 μg/ml of the properdin protein.

20. The method of claim 11, wherein the effective amount of the pharmaceutical composition is selected to achieve a target serum concentration of properdin in the subject in the range of 1-100 μg/ml of the properdin protein.
